(19) United States
(12) Patent Application Publication
(10) Pub. No.: US 2009/0176286 A1
(43) Pub. Date: Jul. 9, 2009

(54) Process for Fractionating Lignocellulosic Biomass into Liquid and Solid Products

(76) Inventors: Ryan P. O'Connor, Broomfield, CO (US); Robert Wooley, Golden, CO (US); Jeffrey John Kolstad, Wayzata, MN (US); Robert Thomas Kean, Minneapolis, MN (US); David A. Glassner, Littleton, CO (US); Beth Mastel, Excalsior, MN (US); Jon Michael Ritzenthaler, Lakeville, MN (US); Robert Henry Birk, Midland, MI (US); Jeffrey Warwick, Waconia, MN (US); James R. Hettenhaus, Charlotte, NC (US); Rebecca Kerin Brooks, Afton, MN (US)

Correspondence Address: GARY C. COHN, PLLC, P. O. Box 313, Huntingdon Valley, PA, 19006, US

(21) Appl. No.: 12/093,630

(22) PCT Filed: Nov. 15, 2006
(86) PCT No.: PCT/US06/44262

§ 371 Date: Jul. 9, 2008

Related U.S. Application Data
(60) Provisional application No. 60/739,854, filed on Nov. 23, 2005.

(51) Int. Cl. C12P 7/56; C12P 7/10; C07G 1/00; C07H 1/08

(52) U.S. Cl. 435/139; 435/165; 530/500; 536/128

(57) ABSTRACT

The invention herein is an efficient, flexible biomass fractionation process comprising digesting a lignocellulosic-biomass material at about 120-220° C. and a pH of less than about 4, in an aqueous mixture containing an effective concentration of at least one solvent for lignin, and separating to recover a solid phase that contains a large fraction of the cellulose originally in the starting lignocellulosic material and a liquid phase that contains most of the lignin and hemicellulose originally in the starting lignocellulosic biomass. The process can produce a solid phase that contains at least 75% cellulose and less than 10% lignin. The cellulose-rich solid product can be converted very efficiently to glucose. The solid product can also be used in commercial pulp applications, such as papermaking or fluff pulp. Hemicellulose sugars and lignin can be used directly or converted to other products.

This application claims benefit of U.S. Provisional Patent Application No. 60/739,854, filed 23 Nov. 2005.

This invention was made with Government support under Cooperative Agreement DE-FC36-03GO13145 awarded by the United States Department of Energy. The Government has certain rights in this invention.

This invention relates to fractionation processes for converting biomass into recoverable cellulose, lignin, and fermentable sugars.

Lignocellulosic biomass is the most abundant renewable material on the planet and has long been recognized as a potential feedstock for producing chemicals, fuels, and materials. Lignocellulosic biomass normally comprises primarily cellulose, hemicellulose, and lignin. Cellulose and hemicellulose are natural polymer mixtures of hexose and pentose sugars, and lignin is an aromatic/aliphatic hydrocarbon polymer reinforcing the entire biomass network. Some forms of biomass do not contain hemicellulose.

There are many reasons why it would be beneficial to process biomass in a way that effectively separates the major fractions (cellulose, hemicellulose, and lignin) from each other. Biomass refining (or “biorefining”) can be viewed as a way to fractionate lignocellulosic biomass into its primary components, and then can be purified or further reacted—by chemical or biochemical routes—to produce higher-value products.

Cellulose from biomass can be used in industrial applications, such as to make paper or other pulp-derived products. The cellulose can also be subjected to further processing to either modify the cellulose in some way or convert it into glucose. Hemicellulose can be hydrolyzed to form its constituent sugars (referred to herein as “hemicellulose sugars”). These hemicellulose sugars can be fermented to a variety of products, such as ethanol or lactic acid, or converted to other chemicals. Lignin from biomass has value as a solid fuel and also as an energy feedstock to produce liquid fuels, synthesis gas, or hydrogen. Lignin is also useful as an intermediate to make a variety of polymeric compounds. Additionally, minor components such as proteins or rare sugars can be extracted and purified for specialty applications.

In light of this objective, a major shortcoming of previous biomass fractionation technologies is that one or two of the major components can be economically recovered in high yields, but not all three. Either the third component is sacrificially degraded in an effort to produce the other two components, or poor fractionation is accomplished. An important illustration of this is traditional biomass pulping (to produce paper and related goods). Cellulose is recovered in high yields, but lignin is primarily consumed by oxidation and hemicellulose sugars are mostly degraded to compounds such as furfural. Approximately half of the starting biomass is essentially wasted in this manufacturing process. Biomass-pretreatment approaches typically can produce high yields of hemicellulose sugars but suffer from moderate cellulose and lignin yields.

One of the most challenging technical obstacles in biomass fractionation processes is that the recovered cellulose is often resistant to subsequent hydrolysis to form glucose. The hydrolysis is often done enzymatically, and the resistance of the cellulose to hydrolyze is often compensated for by using high concentrations of the enzymes. The enzyme cost can be a tremendous burden on the overall cost to turn cellulose into glucose for fermentation. Cellulose can be made to be reactive by subjecting biomass to severe chemistry, but that would jeopardize not only its integrity for other potential uses but also the yields of hemicellulose and lignin.

What is desired is to efficiently fractionate any lignocellulosic-based biomass into its primary components (cellulose, hemicellulose sugars, and lignin) so that each of these components can be recovered in good yield and in a condition that allows them to be used easily in potentially distinct downstream processes. The process preferably has sufficient processing flexibility to permit optimization of product mix. An especially flexible fractionation technique would not only separate most of the hemicellulose and lignin from the cellulose, but also render the cellulose highly reactive to cellulase enzymes for the manufacture of glucose.

This invention is a biomass fractionation process comprising digesting a starting lignocellulosic-biomass material at a temperature of about 120-220° C. and a pH of less than about 4 in an aqueous extractant mixture containing an effective concentration of a solvent for lignin, to form (1) one or more liquid phases that collectively contain dissolved lignin and, if hemicellulose is present in the starting lignocellulosic-biomass material, hemicellulose in dissolved or hydrolyzed form and (2) a solid phase containing cellulose. The liquid and solid phases are separated to recover a solid phase that contains at least 75% of the cellulose originally in the starting lignocellulosic material and one or more liquid phases that contains at least 50% of the lignin originally in the starting lignocellulosic material and, if hemicellulose is present in the starting biomass, at least 50% of the hemicellulose originally in the starting lignocellulosic biomass, in hydrolyzed or dissolved form.

This process permits the efficient fractionation of lignocellulosic-based biomass into its primary major components (cellulose, lignin, and if present, hemicellulose) so that each can be used in potentially distinct processes. An advantage of the process is that it produces cellulose-rich solids while concurrently producing a liquid phase containing a high yield of both hemicellulose sugars and lignin, and low quantities of products formed by the degradation of lignin and hemicellulose sugars.

The process of the invention is a flexible fractionation technique which produces products, particularly cellulose, that are suitable for multiple uses. The technique separates most of the hemicellulose and lignin from the cellulose, resulting in cellulose of good purity. The cellulose is often highly reactive to cellulase enzymes, which allows it to be efficiently converted to glucose.

The process may be conducted batch-wise or continuously, or some combination thereof. Solid and liquid may flow cocurrently or countercurrently, or in any other flow pattern that achieves the desired fractionation.

In a preferred embodiment, the separation of liquid and solid is conducted at a temperature of at least about 120° C. and under superatmospheric pressure such that the water and the solvent for lignin do not boil. In another preferred embodiment, the recovered solid phase is washed with water, a solvent for lignin, or both.

In another preferred embodiment, the extractant mixture has a pH of about 1.5-3.5, more preferably about 2.0-3.0.

In some embodiments in which multiple liquid phases are present, a liquid phase is recovered that contains at least 50% of the lignin originally in the starting lignocellulosic material and at least one other liquid phase is recovered that contains at least 50% of the hemicellulose originally in the starting lignocellulosic material, the hemicellulose in the other liquid phase being in hydrolyzed or dissolved form.

The process produces a solid phase that contains at least 75%, preferably at least 85% by dry weight cellulose and less than 10%, preferably less than 5% by dry weight lignin.

In some embodiments, the process produces a cellulose-rich solid product which can be converted to glucose with at least 80% yield within 24 hours at 50° C. and 2 wt % solids, in the presence of a cellulase enzyme mixture in an amount of no more than 15 filter paper units (FPU) per g of the solid product. In preferred embodiments, this same conversion requires no more than 5 filter paper units (FPU) per g of the solid product. In especially preferred embodiments, the solid product can be converted to glucose with at least 90% yield within 24 hours at 50° C. and 2 wt % solids, in the presence of a cellulase enzyme mixture in an amount of no more than 15 filter paper units (FPU) per g of the solid product.

The process may further comprise drying the solid phase to produce a material having a liquid-absorption capacity of at least 13 g water per g dry solid. The process may also further comprise recovering lignin from a liquid phase. The process may also include the additional step of fermenting the hemicellulose sugars into a desirable fermentation product, such as ethanol, D-lactic acid, L-lactic acid, or a mixture of two or more thereof.

In preferred embodiments, the solvent for lignin is ethanol. In other preferred embodiments, the extractant mixture contains sulfuric acid. An especially preferred extractant mixture includes 20-50% ethanol by weight, and sufficient sulfuric acid to provide a pH of 1.5-3.5 and especially of 2.0 to 3.0.

In the present invention, lignocellulosic biomass is fractionated into its primary components to produce fermentable sugars, cellulose fibers, and lignin.

As used herein, “lignocellulosic biomass” means any material containing cellulose and lignin. Lignocellulosic biomass may also contain hemicellulose. Examples of lignocellulosic-biomass materials include, but are not limited to, corn stover, corn fiber, wheat straw, rice straw, sugarcane bagasse, hardwoods, softwoods, pulp and paper wastes, recycled paper, forest residues, and process streams containing any of these materials. Mixtures of one or more types of lignocellulosic biomass can also be used. In general, the biomass is in the form of a particulate, but particle size is not critical.

In this process, the lignocellulosic biomass is digested with an extractant mixture containing water and a solvent for lignin. The solvent is present at a concentration at least sufficient to extract lignin from the biomass and retain the extracted lignin in solution. The pH of the extractant mixture is about 4 or less. Water hydrolyzes the hemicellulose, if present, to simple sugars such as xylose and oligomers thereof. Water is also believed to play a role in breaking down the biomass structure to release lignin from the starting material. The extractant mixture contains at least the total amount of water needed for the desired overall reaction stoichiometry, but more can be used as long as the lignin is solubilized in the extractant mixture. There are practical advantages to using high concentrations of water in the extractant mixture, including the low cost of water relative to other solvents and the benefit of having excess water available to increase both hydrolysis rates and drive the hydrolysis of the hemicellulose sugars more strongly to form monomeric rather than oligomeric sugars. Preferably, the process operates with about 10-90 wt % water in the liquid phase. The optimal water concentration will be influenced by the lignin content of the starting material and by the total liquid/solid ratio of the process. It is desired economically to keep the liquid/solid ratio as low as possible, which favors using a water concentration of 10 to 90 wt %, preferably from 25 to 85 wt % and more preferably from about 50 to 80 wt % water, in the extractant mixture, based on the combined weight of water and solvent.

A solvent for lignin is also present in the extractant mixture. A wide variety of solvents can be employed effectively, provided the lignin is soluble in the extractant mixture at process conditions. Preferably, enough solvent is included in the extractant mixture to dissolve the lignin present in the starting material. The solvent for lignin may be completely miscible with water, in which case there will be only one liquid phase. In that case, mass transfer of lignin and hemicellulose into the liquid phase is enhanced, and the separation step must only deal with one liquid stream. Alternatively, the solvent for lignin may be immiscible with water or only partially miscible with water, in which case more than one liquid phase may form. When the solvent is immiscible or only partially miscible in water, the extractant mixture readily separates to form liquid phases, so a subsequent step to separate the solvent from the water can be avoided or simplified. This can be advantageous if one liquid phase contains most of the lignin and the other contains most of the hemicellulose sugars, as this facilitates recovering the lignin from the hemicellulose sugars.

Ethanol is one preferred solvent due to cost and its effectiveness as a solvent for lignin. A suitable range of ethanol concentrations in the liquid phase is between about 10-90 wt %, preferably about 15-75 wt % ethanol, and more preferably about 20-50 wt %. Concentrations of ethanol greater than about 50 wt % tend to be less favorable economically, due in part to the higher cost of ethanol relative to water and the high vapor pressure of ethanol which increases equipment costs and safety considerations.

Other suitable solvents include linear aliphatic alcohols such as methanol, 1- or 2-propanol, glycerol, 1- or 2-butanol; cyclic aliphatic alcohols such as cyclohexanol; aromatic alcohols such as phenol or cresols; ketones such as cyclohexanone or methyl ethyl ketone; or ethers such as diethyl ether (ethoxyethane). It is envisioned that a wide variety of alcohols, ketones, or ethers are useful in the process. These other solvents can be used in proportions as described above with respect to ethanol.

Protons in solution enhance the rates of both the hydrolysis of hemicellulose, if present, and the extraction of lignin. The extractant mixture therefore has a pH of less than about 4.0 during the fractionation process. pH is measured for this purpose by removing a sample of the extractant mixture from the process and cooling it to about room temperature (22° C.). The preferred pH is between about 1.5 and about 3.5, more preferably between about 2.0 and about 3.0. The desired pH is chosen in conjunction with time and temperature, because all three factors impact fractionation rates. Generally, longer times and/or higher temperatures are necessary when the pH is in the higher end of the aforementioned ranges. It is preferable, but not necessary, for the pH of the extractant mixture at the start of the fractionation process (prior to heating up the mixture) to be less than 4.0. In most cases, the pH of the extractant mixture can be adjusted to within the aforementioned ranges by adding a protic acid. Preferably, strong acids such as sulfuric, sulfurous, hydrochloric, or trifluoroacetic acid are added, although weaker organic acids such as lactic, citric, glycolic, or propionic acid can also be added.

The specific concentration of acid needed (if any) to obtain a given desired pH depends in part on the pKa of the acid. The buffering capacity of the particular lignocellulosic-biomass feedstock also can affect the amount of acid that is needed, as some lignocellulosic-biomass materials can release components during the digestion process which are bases or buffers that can raise the pH somewhat. Acid addition during the process can be used to maintain the desired pH, if desired.

It is also possible to obtain the desired pH by instead adding to the extractant mixture a precursor that dissociates or otherwise reacts in situ to generate an acid. A preferred type of precursor is a carboxylic acid ester, which can dissociate to form an acid and an alcohol. The alcohol can function as all or part of the solvent for lignin, as can any quantity of the ester that does not dissociate. A preferred precursor of this type is ethyl lactate, which will be partially converted to ethanol and lactic acid in aqueous solution.

A suitable concentration of solids in the reacting mixture is between about 1 wt % and about 50 wt % solids, depending on equipment configuration. Preferably, the weight ratio of extractant mixture to starting biomass is from about 4:1 to about 25:1, especially from about 9:1 to about 19:1. The biomass can be impregnated with the extractant mixture or any component thereof for a certain period of time, such as one hour, prior to heating up the mixture to the fractionation temperature. This step enhances the uniformity of the solid-liquid slurry prior to the fractionation process, and may slightly decrease the digestion time needed. The extent of time for impregnation, if any, is not regarded as critical.

The biomass is digested in the extractant mixture at a temperature of from about 120° C. to about 220° C. 120° C. is approximately the glass-transition temperature of lignin, below which lignin is very difficult to extract at reasonable rates. Above 220° C., side reactions become so fast (regardless of pH) that the process becomes difficult to control. Those side reactions include, for example, degradation of hemicellulose sugars (if present) to furfural, lignin polymerization and precipitation, and complex formation between lignin and other components in solution. Temperatures between 170° C. and 190° C. generally offer a good kinetic balance between the rates of desired and undesired reactions. The digestion is conducted under superatmospheric pressure such that the water and solvent for lignin do not boil.

A suitable time period for the digestion step is between about 1 minute and about 24 hours, preferably between about 5 minutes and about 2 hours, more preferably between about 10 minutes and about 1 hour. Generally, there is an inverse relationship between the temperature used during the digestion step and the time needed to obtain good fractionation of the biomass into its constituent parts. Also, less time may be needed when smaller biomass particles are fed to the reactor, due to faster mass transfer of the aqueous mixture into and out of the solid phase.

Once the desired amount of fractionation of both hemicellulose and lignin from the solid phase is achieved, the liquid and solid phases are separated. Conditions for the separation are preferably selected to minimize the reprecipitation of the extracted lignin on the solid phase. This is favored by conducting the separation step at a temperature of at least the glass-transition temperature of lignin (about 120° C.). When the separation is conducted above the boiling temperature of water and/or the solvent, the separation is conducted under pressure to prevent the water and/or solvent from flashing. It is preferred to cool the liquid phase down below about 50° C. quickly after it is removed from the solid mass.

It is within the scope of the invention to cool the digested mass to below the lignin glass-transition temperature prior to separating the solid and liquid phases, but this tends to lead to a greater amount of the lignin being retained on the solid-phase materials.

The physical separation can be accomplished either by transferring the entire mixture to a device that can carry out the separation and washing, or by removing only one of the phases from the reactor while keeping the other phase in place. The solid phase can be physically retained by appropriately sized screens through which liquid can pass. The solid is retained on the screens and can be kept there for successive solid-wash cycles. Alternately, the liquid could be retained and solid phase forced out of the reaction zone, with centrifugal or other forces that can effectively transfer the solids out of the slurry. In a continuous system, countercurrent flow of solids and liquid can accomplish the physical separation.

The recovered solids normally will contain a quantity of lignin and sugars (including hemicellulose sugars), some of which is redeposited during the separation step and can be removed easily by washing. The washing-liquid composition can be the same as or different than the solvent composition used during fractionation. Multiple washes can be performed to increase effectiveness. Preferably, one or more washes are performed with a composition including a solvent for lignin, to remove additional lignin from the solids, followed by one or more washes with water to displace residual solvent and sugars from the solids. Recycle streams, such as from solvent-recovery operations, can be used to wash the solids.

After separation and washing as described, a solid phase and at least one liquid phase are obtained. The solid phase contains substantially undigested cellulose. A single liquid phase is usually obtained when the solvent and the water are miscible in the relative proportions that are present. In that case, the liquid phase contains, in dissolved form, most of the lignin originally in the starting lignocellulosic material, as well as soluble monomeric and oligomeric sugars formed in the hydrolysis of any hemicellulose that may have been present. Multiple liquid phases tend to form when the solvent and water are wholly or partially immiscible. The lignin tends to be concentrated in the liquid phase that contains the highest concentration of the solvent. Hemicellulose sugars tend to be present in the liquid phase that contains the highest concentration of water.

At least 50%, preferably at least 75%, more preferably at least 90% by weight of the lignin in the starting biomass is extracted into the liquid phase(s). At least 80%, preferably at least 90%, more preferably at least 95% by weight of the cellulose in the starting biomass remains in the solids. Generally, between 50% and 90% of the hemicellulose (if any) in the starting biomass is hydrolyzed and extracted into the liquid phase. It is preferable to hydrolyze and dissolve at least 75%, more preferably at least 85% of the starting hemicellulose into the liquid phase.

The solid phase will preferably contain at least 65 dry wt %, preferably at least 75 dry wt %, more preferably at least 85 dry wt % of cellulose. The lignin content of the solid product is less than 20 dry wt %, preferably less than 10 dry wt %, and more preferably less than 5 dry wt %. The hemicellulose content of the solid product is less than 30 dry wt %, preferably less than 15 dry wt %, and more preferably less than 5 dry wt %. The dried solid-phase material may in addition contain small quantities of proteins, uronic acids, ash, and dirt.

The liquid phase(s) contains most of the lignin in dissolved form, and most of the hemicellulose (if present) typically in the form of hemicellulose sugars. The predominate hemicellulose sugars are xylose or arabinose, or their respective oligomers, depending on the type of lignocellulosic biomass used in the process. An advantage of the invention is that it is possible to obtain good yields of hemicellulose sugars with little further degradation of those products to less-valuable species. In the most-preferred case, in which hemicellulose is hydrolyzed to xylose, the formation of the xylose degradation product, furfural, is minimized. The percentage of hemicellulose in the starting biomass that is hydrolyzed but then degraded to furfural is preferably less than 10%, more preferably less than 5% by weight.

Generally, the cellulose-rich solid product can be used in industrial cellulose applications directly, with or without drying, or subjected to further processing to either modify the cellulose in some way or convert it into glucose.

The cellulose-rich solid product can be processed into paper products any convenient methods (Macdonald, Papermaking and Paperboard Making, Vol. 3, TS 1105.J66, 1969). The solid product is also useful as fluff pulp, which is commonly used in absorbent applications such as diapers and consumer wipes.

Cellulose recovered from the solid phase is particularly suitable for manufacturing dissolving pulp (also known as □-cellulose), when its purity is 85% by weight or more. In some cases, cellulose of that purity is obtained simply by washing and drying the separated solid phase. If needed, the recovered cellulose can be further purified using various techniques, such as bleaching. Cellulose having a purity of 95 wt % or more can be obtained in this manner.

The cellulose obtained in the process of the invention in most cases is easily and rapidly hydrolyzed to glucose and soluble glucose oligomers. Although the invention is not limited to any theory, it is believed that the resistance of cellulose from biomass fractionation processes to enzymatic hydrolysis is due at least in part to the presence of lignin on the surface of the cellulose fibers. The lignin is believed to form a physical barrier to water, thus causing the hydrolysis to proceed slowly. In this invention, the efficient removal of lignin exposes more cellulose at the surface of the fibers, allowing better contact with water (and added enzymes or other catalyst), and therefore increasing the rate of reaction. The overall yield of glucose can generally be at least 90% of the potential glucose contained in the biomass feedstock. The ease of which cellulose can be hydrolyzed to glucose and soluble oligomers can be expressed in terms of the amount of glucose that is produced within a given time under specific hydrolysis conditions. In preferred embodiments, glucose yields exceeding 90% can be obtained from the washed cellulose produced in accordance with the invention in less than 24 hr using 5 filter paper units (FPU, a measure of enzyme activity) cellulase enzyme per g cellulose, when assayed according to Brown and Torget, “Enzymatic Saccharification of Lignocellulosic Biomass,” NREL Laboratory Analytical Procedure LAP-009, August 1996. The glucose so produced can be used as a fermentation substrate to produce for example lactic acid, citric acid, ethanol, or amino acids. It can be used as a sweetener or isomerized to enrich its fructose content. The glucose can be used to produce baker's yeast. The glucose can be catalytically or thermally converted to various organic acids and other materials.

A lignin product can be readily obtained from a liquid phase using one or more of several methods. One simple technique is to evaporate off all liquid, resulting in a solid lignin-rich residue. This technique would be especially advantageous if the solvent for lignin is water-immiscible. Another method is to cause the lignin to precipitate out of solution. Some of the ways to precipitate the lignin include (1) removing the solvent for lignin from the liquid phase, but not the water, such as by selectively evaporating the solvent from the liquid phase until the lignin is no longer soluble; (2) diluting the liquid phase with water until the lignin is no longer soluble; and (3) adjusting the temperature and/or pH of the liquid phase. Methods such as centrifugation can then be utilized to capture the lignin. Yet another technique for removing the lignin is continuous liquid-liquid extraction to selectively remove the lignin from the liquid phase, followed by removal of the extraction solvent to recover relatively pure lignin.

Lignin produced in accordance with the invention can be used as a fuel. As a solid fuel, lignin is similar in energy content to coal. Lignin can act as an oxygenated component in liquid fuels, to enhance octane while meeting standards as a renewable fuel. The lignin produced herein can also be used as polymeric material, and as a chemical precursor for producing lignin derivatives.

When hemicellulose is present in the starting biomass, all or a portion of the liquid phase contains hemicellulose sugars, which include monomeric sugars and soluble oligomeric sugars. These sugars can be fermented in a subsequent operation to form a variety of useful fermentation products. It is preferred in such cases to remove most of the lignin from the liquid, as described above, to produce a fermentation broth which will contain water, possibly some of the solvent for lignin, hemicellulose sugars, and various minor components from the digestion process. This fermentation broth can be used directly, combined with one or more other fermentation streams, or further treated. Further treatment can include sugar concentration by evaporation; addition of glucose or other sugars (optionally as obtained from cellulose saccharification described above); addition of various nutrients such as salts, vitamins, or trace elements; pH adjustment; and removal of fermentation inhibitors such as acetic acid and phenolic compounds. The choice of conditioning steps should be specific to the target product(s) and microorganism(s) employed.

The sugar profile of the liquid phase will depend on the specific type of biomass fed to the process. The major hemicellulose sugar in solution is usually xylose (from hardwoods, corn stover, etc.) or arabinose (from e.g. softwoods), which are both five-carbon sugars that can be fermented to lactic acid, ethanol, and other chemicals, depending on the biocatalyst used. Examples of processes and yeast species for fermenting a xylose-containing fermentation medium to fermentation products such as ethanol and lactic acid are described, for example, in US Published Patent Application 2004/0142456, WO 2004/067760 and WO 2004/085627. Xylose can also be converted into xylitol.

The following examples are provided to illustrate the invention, but are not intended to limit the scope thereof. All parts and percentages are by weight unless otherwise indicated.

### EXAMPLE 1

Corn stover is chopped and washed with water to remove dirt and fine particles. The composition of the chopped and washed corn stover is about 37 wt % cellulose (glucan), 22 wt % hemicellulose (xylan), 22 wt % lignin, and 19 wt % other materials.

The corn stover is soaked in the process solvent at a liquid/solid ratio of 15:1 (by weight) for 1 hr at room temperature, and then fed into a reactor. The extractant mixture in this example is about 70 wt % water, about 30 wt % ethanol, and 0.2 wt % (approximately 0.04 N) sulfuric acid. The initial solution pH is about 2.0.

The mixture is heated to 170° C. in an agitated, pressurized reaction vessel. After 30 minutes at 170° C., the entire contents of the reactor are quickly transferred out of the high-temperature reaction zone into a pressurized separation zone at approximately 130° C. Nitrogen gas is introduced under a pressure of ˜5 atm to rapidly press the liquid from the solids. This allows the cellulose-rich solids to be separated from the liquid phase in about 30 seconds. The solids are washed by feeding 0.5 L of ethanol into the filtration vessel, followed by 5-atm nitrogen pressing to remove the wash liquid from the solids. This ethanol wash is performed five consecutive times, followed by five water washes of 0.5 L each. During each washing, the filtration vessel is maintained at approximately 130° C. The ethanol washing further removes lignin from the solids, while the water washing removes residual water-soluble species as well as residual solvent. Separate tanks are used to receive (1) the primary filtrate from the initial separation, (2) the cumulative ethanol washate, and (3) the cumulative water washate.

Approximately 60% of the initial corn stover solids are solubilized during this fractionation process. Approximately 92% of the cellulose initially in the corn stover remains in the solid phase. 91% of the initial hemicellulose is contained in the liquid phase in the form of xylose and soluble xylose oligomers, and 85% of the initial lignin is dissolved in the liquid phase. The yield of fermentable xylose in the hydrolyzate is about 80% of the xylan present in the initial corn stover. The composition of the solids after drying at about 90° C. for 24 hours is approximately 83 wt % cellulose, 5 wt % hemicellulose, 7 wt % lignin, and 5 wt % other materials.

### EXAMPLE 2

A mixture of corn stover and process solvent are prepared in a similar way as in Example 1. The solvent composition in this example is about 50 wt % water, about 50 wt % ethanol, and 0.2 wt % sulfuric acid. The initial liquid/solid ratio is 25:1 (by weight). The initial solution pH is about 2.0. The mixture is reacted at 170° C. for 10 min before being separated and washed as described in Example 1.

The washed solid phase is further digested with cellulase enzymes to produce glucose monomer, as follows. A shake flask is loaded with the solid product at about 2 wt % solids and 15 FPU (filter-paper units) per g glucan of Spezyme CP (Genencor) with 10%, on a protein basis, of Novozym-188 α-Glucosidase (Novozymes), according to the analytical procedure published in Brown and Torget, “Enzymatic Saccharification of Lignocellulosic Biomass,” NREL Laboratory Analytical Procedure LAP-009, August 1996. Cellulose saccharification is carried out at 50° C. Only about 7 hr are necessary to reach 80% conversion to glucose, and the cellulose conversion to glucose is 95% after 24 hours.

### EXAMPLE 3

Raw, ground corn stover is physically segregated into three particle sizes with a Ro-Tap classifier: long fibers (greater than about 1 mm), short fibers (between about 0.1 mm and about 1 mm), and fines (less than about 0.1 mm).

The short corn-stover fibers are mixed with process solvent having the composition of about 50 wt % water, about 50 wt % ethanol, and 0.3 wt % sulfuric acid. The initial solution pH is about 1.5.

The solid feed is soaked in the process solvent at a liquid/solid ratio of 25:1 (by weight) for 1 hr at room temperature, and then the entire mixture is reacted for 10 min at 170° C. under pressure. The reactor contents are then quickly transferred into a pressurized separation zone at approximately 130° C., and subsequently washed with ethanol and then with water, as described in Example 1.

Approximately 61 wt % of the corn stover is solubilized by the process. The composition of the solid phase after drying at 90° C. for 24 hours is approximately 75 wt % cellulose, 4 wt % hemicellulose, 7 wt % lignin, and 14 wt % other materials. Approximately 86% of the cellulose contained in the corn stover remains in the solid phase. 93% of the hemicellulose (in the form of xylose and soluble hydrolysis products), and 86% of the lignin contained in the corn stover remain in the liquid-phase product. The yield of fermentable xylose in the liquid phase is about 80% of the xylan present in the corn stover feed.

The long fibers are soaked in the process solvent and fed into the reactor. The solvent composition in this example is about 70 wt % water, about 30 wt % ethanol, and 0.2 wt % sulfuric acid. The initial liquid/solid ratio is 5:1 (by weight). The initial solution pH is about 2.0. The mixture is reacted at 170° C. for 30 min before being separated and washed as described in Example 1.

The washed and dried (90° C. for 24 hours) cellulose-rich product is subjected to a liquid-absorbency test in which water is allowed to absorb into the solid fibers until equilibrium is reached. The liquid uptake ratio, by mass, in this example is about 13 g/g.

### EXAMPLE 4

A mixture of corn stover and process solvent are prepared in the manner described in Example 1. The solvent composition in this example is about 60 wt % water, about 40 wt % ethanol, and 0.1 wt % sulfuric acid. The initial solution pH is about 3.0. The initial liquid/solid ratio is 20:1 (by weight). The mixture is reacted at 180° C. for 20 min prior to being separated and washed as described in Example 1.

Approximately 59% of the corn-stover solids become solubilized. The composition of the solid phase (after washing and drying at 90° C. for 24 hours) is approximately 80 wt % cellulose, 7 wt % hemicellulose, 7 wt % lignin, 6 wt % other materials. Approximately 89% of the cellulose contained in the starting material remains in the solid phase. 87% of the initial hemicellulose (as hemicellulose sugars), and 87% of the initial lignin, are contained in the liquid phase. The yield of fermentable hemicellulose sugars in the liquid phase is about 78% of the hemicellulose present in the corn-stover feed.

### EXAMPLE 5

A mixture of corn stover and process solvent is prepared, in which the solvent composition is about 50 wt % water, about 50 wt % 1-butanol, and 0.1 wt % sulfuric acid. The corn stover is not washed prior to forming the mixture. The initial liquid/solid ratio is 15:1 (by weight). The initial solution pH is about 2.5. The mixture is reacted at 170° C. for 1 hr.

After reaction, the solid-liquid mixture is allowed to cool to room temperature. The solid and liquid are separated by a syringe filter, and the solids are dried for analysis.

The solid phase contains approximately 50 wt % cellulose, 11 wt % hemicellulose, and 10 wt % lignin, with the remainder comprising primarily ash and protein.

### EXAMPLE 6

A mixture comprising corn stover and process solvent is prepared, in which the solvent composition is about 33 wt % water, about 66 wt % ethanol, and about 1 wt % lactic acid. The initial liquid/solid ratio is 10:1 (by weight). The initial solution pH is about 3.8. The mixture is reacted at 200° C. for 1 hr. After reaction, the solid-liquid mixture is allowed to cool to room temperature. The solid and liquid are separated by a syringe filter. The dried solid composition contains approximately 58 wt % cellulose, 15 wt % hemicellulose, and 13 wt % lignin, with the remainder comprising primarily ash and protein.

### EXAMPLE 7

In this example multiple receiving tanks are used to achieve simulated countercurrent flow. This mode simulates the effect of a moving bed of solids against successively cleaner liquids.

A first experiment (run 1) uses process conditions and a solvent composition as described in Example 1, except that the batch time is 10 min. The filtrate mixture is collected (“black liquor”). A second experiment (run 2) returns the reacted biomass from run 1 to the reactor, and using fresh solvent, reacts again for 10 min to produce filtrate called “weak black liquor.” Run 3 begins with the twice-reacted solids and fresh solvent, resulting in “brown liquor.” The liquid preparation is compete, and the weak-black and brown liquors remain heated in separate tanks.

To simulate the first stage of a countercurrent process (run 4), fresh corn stover is digested in the weak-black liquor from run 2. After digesting for 10 min at 170° C., the solids and liquid are separated in the manner described in Example 1 and the solids are returned to the reactor. The liquid phase is the hydrolyzate that contains the hemicellulose sugars to be fermented. Evaporation is used to remove most of the ethanol and some of the water, until a target xylose concentration is achieved (e.g., 80 g/L xylose). The evaporated ethanol/water is recycled to the front of the simulated process as “white liquor,” with appropriate addition of sulfuric acid, if necessary, to reach 0.2 wt % sulfuric acid.

The second stage of a countercurrent process (run 5) is simulated by combining the solids from run 4 with the brown liquor from run 3 above. After digesting for 10 min at 170° C., followed by separation, the filtrate is stored hot. The third stage of a countercurrent process is simulated (run 6) by combining the solids from run 5 along with fresh extraction medium, which is recycled solvent recovered from the black liquor from run 4. As in the other stages, the time and temperature in run 6 are 10 min and 170° C. This final stage produces a cellulose-rich solid product along with brown liquor. The solid phase is subjected to a final water wash to remove residual ethanol. This water washate contains a small amount of ethanol and is combined with recycled ethanol to produce white liquor (30 wt % ethanol, 0.2 wt % sulfuric acid).

1. A biomass fractionation process comprising
a) digesting a starting lignocellulosic-biomass material at a temperature of from about 120° C. to about 220° C. and a pH of less than about 4, in an aqueous extractant mixture containing an effective concentration of a solvent for lignin, to form a digested mass that includes (1) one or more liquid phases that collectively contain dissolved lignin and, if hemicellulose is present in the starting lignocellulosic-biomass material, hemicellulose in dissolved or hydrolyzed form and (2) a solid phase containing cellulose; and
b) separating said liquid and solid phases to recover a solid phase that contains at least 75% of the cellulose originally in the starting lignocellulosic material and a liquid phase that contains at least 50% of the lignin originally in the starting lignocellulosic material and, if hemicellulose is present in the starting lignocellulosic-biomass material, at least 50% of the hemicellulose originally in the starting lignocellulosic material, in the form of hemicellulose sugars.

2. The process of claim 1, wherein said separation is conducted at a temperature of at least about 120° C. and under superatmospheric pressure such that the water and the solvent for lignin do not boil.

3. The process of claim 1 wherein the starting biomass material contains hemicellulose.

4. The process of claim 3, wherein the solvent for lignin is at least partially immiscible with water and the digested mass contains multiple liquid phases.

5. The process of claim 4, further comprising separating multiple liquid phases to recover a liquid phase that contains at least 50% of the lignin originally in the starting lignocellulosic material and at least one other liquid phase that contains at least 50% of the hemicellulose originally in the starting lignocellulosic material, in the form of hemicellulose sugars.

6. The process of claim 5 wherein the solid phase contains at least 75% by dry weight cellulose and less than 10% by dry weight lignin.

7. The process of claim 6 wherein the solid phase contains at least 85% by dry weight cellulose and less than 5% by dry weight lignin.

8. The process of claim 5 wherein the recovered solid phase is washed with water, a solvent for lignin, or both.

9. The process of claim 4 comprising the further step of c) recovering lignin from a liquid phase.

10. The process of claim 3, further comprising d) fermenting the hemicellulose sugars.

11. The process of claim 10, wherein the hemicellulose sugars are fermented to ethanol, D-lactic acid, L-lactic acid, or a mixture of two or more thereof.

12. The process of claim 1 further comprising e) drying the solid phase to produce a material having a liquid-absorption capacity of at least 13 g water per g dry solid.

13. The biomass fractionation process of claim 5 wherein step a) is conducted at a pH between about 1.5 and about 3.5.

14. The biomass fractionation process of claim 5 wherein step a) is conducted at a pH between about 2.0 and about 3.0.

15. The process of claim 1, wherein steps a) and b) are performed continuously and the solid and liquid flow cocurrently.

16. The process of claim 1 wherein steps a) and b) are performed continuously and the solid and liquid flow countercurrently.

17. The process of claim 1, wherein the solvent for lignin is ethanol.

18. The process of claim 1, wherein the solvent for lignin includes at least one of methanol, ethanol, 1- and 2-propanol, glycerol, 1- and 2-butanol, cyclohexanol, phenol, cresol, cyclohexanone, methyl ethyl ketone, and diethyl ether.

19. The process of claim 1 wherein the extractant mixture contains sulfuric acid.

20. The process of claim 1 wherein the extractant mixture contains at least one of sulfuric acid, sulfurous acid, hydrochloric acid, trifluoroacetic acid, lactic acid, ethyl lactate, citric acid, glycolic acid, and propionic acid.

21. The process of claim 1, wherein the extractant mixture contains 50-80 wt % water and 50-20 wt % of said solvent, based on the combined weight of water and solvent, and the extractant mixture has a pH of 1.5 to 3.5.

22. The process of claim 21, wherein step b) is conducted at a temperature of at least 120° C. and under sufficient pressure to keep the water and solvent from boiling.

23. The process of claim 21, wherein the solvent is ethanol and the extractant mixture contains sulfuric acid.

24. The process of claim 23, wherein the solid phase contains less than 5% by weight of hemicellulose.

25. A cellulose-rich solid product having a liquid-absorption capacity of at least 13 g water per g dry solid.

26. A cellulose-rich solid product which can be converted to glucose with at least 80% yield within 24 hours at 50° C. and 2 wt % solids, in the presence of a cellulase enzyme mixture in an amount of no more than 15 filter paper units (FPU) per g of the solid product.

27. The product of claim 26 which can be converted to glucose with at least 80% yield within 24 hours at 50° C. and 2 wt % solids, in the presence of a cellulase enzyme mixture in an amount of no more than 5 filter paper units (FPU) per g of the solid product.

28. The product of claim 27 which can be converted to glucose with at least 90% yield within 24 hours at 50° C. and 2 wt % solids, in the presence of a cellulase enzyme mixture in an amount of no more than 15 filter paper units (FPU) per g of the solid product.

29. A biomass fractionation process comprising
a) digesting a starting lignocellulosic-biomass material at a temperature of from about 160° C. to about 220° C. and a pH of up to 3, in an aqueous extractant mixture containing an effective concentration of a solvent for lignin, to form a digested mass that includes (1) a liquid phases that collectively contains dissolved lignin and-hemicellulose in dissolved or hydrolyzed form and (2) a solid phase containing cellulose; and
b) separating said liquid and solid phases to recover a solid phase that contains at least 90% of the cellulose originally in the starting lignocellulosic material and a liquid phase that contains at least 75% of the lignin originally in the starting lignocellulosic material and at least 75% of the hemicellulose originally in the starting lignocellulosic material, in the form of hemicellulose sugars;
wherein the solvent for lignin includes at least one of methanol, ethanol, 1- and 2-propanol, glycerol, 1-butanol, 2-butanol, cyclohexanol, phenol, cresol, cyclohexanone, methyl ethyl ketone, and diethyl ether;
wherein the extractant mixture contains at least one of sulfuric acid, sulfurous acid, hydrochloric acid, trifluoroacetic acid, lactic acid, ethyl lactate, citric acid, glycolic acid, and propionic acid;
and wherein said separation is conducted at a temperature of at least about 120° C. and under superatmospheric pressure such that the water and the solvent for lignin do not boil.

30. The process of claim 29 wherein the solvent for lignin is ethanol, methanol or a mixture thereof, the extractant mixture contains sulfuric acid, sulfurous acid, hydrochloric acid or a mixture of two or more thereof, and the pH is from 2.0 to 3.0.
